(19) United States
(12) Patent Application Publication
(10) Pub. No.: US 2019/0022101 A1
(43) Pub. Date: Jan. 24, 2019

(54) Treating Cochlear Synaptopathy

(71) Applicant: Massachusetts Eye and Ear Infirmary, Boston, MA (US)

(72) Inventors: Sharon Kujawa, Boston, MA (US); Albert Edge, Brookline, MA (US); M. Charles Liberman, Boston, MA (US)

(73) Assignee: Massachusetts Eye and Ear Infirmary, Boston, MA (US)

(21) Appl. No.: 16/068,260

(22) PCT Filed: Jan. 6, 2017
(86) PCT No.: PCT/US17/12527

§ 371 Date: Jul. 5, 2018

Related U.S. Application Data
(60) Provisional application No. 62/275,626, filed on Jan. 6, 2016.

(51) Int. Cl. A61K 31/55; A61K 31/135; A61K 31/166; A61K 31/352; A61P 27/16; A61K 38/12

(52) U.S. Cl. CPC A61K 31/55; A61K 31/135; A61K 31/166; A61K 9/0053; A61P 27/16; A61K 38/12; A61K 31/352

(57) ABSTRACT

Methods of treating or reducing the risk of developing hidden hearing loss by administering a small molecule Trk agonists (e.g., amitriptyline, imipramine, LM 22A4 (N,N′,N″Tris(2-hydroxyethyl)-1,3,5-benzenetricarboxamide), 7, 8-dihydroxyflavone (DHF), 7,8,3′-Trihydroxyflavone (THF), Mab2256, neurotrophin-4 (NT-4), neurotrophin-3 (NT-3), brain derived neurotrophic factor (BDNF), nerve growth factor (NGF), N-acetylserotonin, N-[2-(5-Hydroxy-1H-indol-3-yl)ethyl]-2-oxo-3-piperidinecarboxamide (HIOC), deoxygedunin, LM-22A4, or tricyclic dimeric peptide 6 (TDP6)).

### CLAIM OF PRIORITY

This application claims the benefit of U.S. Provisional Patent Application Ser. No. 62/275,626, filed on Jan. 6, 2016. The entire contents of the foregoing are hereby incorporated by reference.

### FEDERALLY SPONSORED RESEARCH OR DEVELOPMENT

This invention was made with Government support under Grant Nos. RO1 DC08577, RO1 DC007174, RO1 DC0188, P30 DC05209, awarded by the National Institutes of Health and Grant No. W81XWH-15-0103 awarded by the Department of Defense. The Government has certain rights in the invention.

### TECHNICAL FIELD

At least in part, the invention relates to methods of treating or reducing the risk of developing hidden hearing loss by administering a small molecule Trk receptor agonist (e.g., a TrkA, TrkB and/or TrkC agonist such as amitriptyline, imipramine, LM 22A4 (N,N′,N″Tris(2-hydroxyethyl)-1,3,5-benzenetricarboxamide), 7,8-dihydroxyflavone (DHF), 7,8,3′-trihydroxyflavone (THF), Mab2256, neurotrophin-4 (NT-4), neurotrophin-3 (NT-3), brain derived neurotrophic factor (BDNF), nerve growth factor (NGF), N-acetylserotonin, N-[2-(5-Hydroxy-1H-indol-3-yl)ethyl]-2-oxo-3-piperidinecarboxamide (HIOC), deoxygedunin, LM-22A4, or tricyclic dimeric peptide 6 (TDP6)) before, during or after noise exposure.

### BACKGROUND

The inner hair cell (IHC)-cochlear nerve fiber synapse is the primary conduit through which information about the acoustic environment is transmitted to the auditory nervous system. In the normal ear, 95% of cochlear nerve fibers make synaptic connection only with IHCs (Spoendlin H (1972). Acta Otolaryngol 73:235-248). Each cochlear nerve fiber has a cell body in the spiral ganglion, a peripheral axon in the osseous spiral lamina and an unmyelinated terminal dendrite in the organ of Corti, with a terminal swelling that forms a synapse with the IHC. The synapse is comprised of a presynaptic ribbon surrounded by a halo of neurotransmitter-containing vesicles (Nouvian et al. (2006). J Membrane Biol. 209:153-165), and a postsynaptic active zone on the cochlear nerve terminal, with glutamate (AMPA-type) receptors for the released neurotransmitter (Matsubara et al. (1976). J Neurosci. 16:4457-4467; Ruel et al. (2007). Hear Res. 227:19-27). The capabilities of this synapse push biological limits in their ability to convey graded temporal information about the stimulus and in maintaining this temporal coding fidelity over a large dynamic range (Moser et al. (2006). J Physiol 576.1:55-62). Loss of these synaptic communications would thus be expected to have significant consequences for function.

This loss has been termed ‘hidden hearing loss’ (Schaette and McAlpine (2011). J. Neuroscience. 31(38):13452-13457), because synaptic and neural losses of less than about 80-90% are not revealed by standard, threshold-based assessments of function (Kujawa and Liberman (2015). Hear Res. 2015 Mar. 11. pii: S0378-5955(15)00057-X. doi: 10.1016/j.heares.2015.02.009). Such losses also are not documented by traditional light microscopy, which does not render the synapses or the unmyelinated terminals of cochlear neurons visible. They are nevertheless common, occurring in ears with permanent threshold elevation as well as in those without (Kujawa et al. (2006). J. Neurosci. 26(7):2115-2123). We now know that IHC-cochlear neuron synapses are primary targets of noise exposure and aging (Kujawa and Liberman (2009). J Neurosci. 29(45):14077-14085; Sergeyenko et al. (2013). J Neurosci. 33(34):13686-13694; Fernandez et al. (2015). J Neurosci. 35(19):7509-7520), two common causes of hearing loss in humans, and their loss likely plays a primary role in other forms of acquired hearing loss as well (Liu et al. (2013) Mol Neurobiol. December 48(3):647-54; Liberman et al. (2015) PLoS ONE 10(11):e0142341. Doi:10.1371/journal.pone.0142341). Although synapses and cochlear nerve terminals can be lost soon after exposure, loss of the neurons themselves is delayed and can progress for months to years. This delay provides a therapeutic window of opportunity to regenerate neurons and their synaptic connections with intact hair cells to treat or prevent hidden hearing loss.

### SUMMARY

The present disclosure is based, at least in part, on the method of treating or reducing the risk of developing hidden hearing loss (HHL) through the use of a small molecule Trk agonist. In some aspects, this disclosure provides a method of treating or reducing the risk of developing hidden hearing loss (HHL) in a subject, e.g., an aging subject or one who will be exposed to noise or ototoxic drugs, e.g., a permanent threshold shifting (PTS) or temporary threshold-shifting (TTS) exposure, the method comprising administering to the subject a therapeutically effective amount of a small molecule Trk agonist, e.g., a TrkB and/or TrkC agonist, wherein the method comprises administering one dose up to 12 hours before an episode of noise exposure, and/or optionally one or more doses after the end of the episode of noise exposure, e.g., at least one dose within 6 to 12 or 24 hours after termination of the noise.

In some aspects, this disclosure provides for the use of a small molecule Trk agonist, e.g., a TrkB and/or TrkC agonist, for treating or reducing the risk of developing HHL in a subject who will be exposed to a temporary threshold-shifting (TTS) noise, wherein the small molecule therapeutic is administered in one dose up to 12 hours before an episode of noise exposure, and/or optionally one or more doses after the end of the episode of noise exposure, e.g., at least one dose within 6 to 12 or 24 hours after termination of the noise.

In some embodiments of all aspects, this disclosure provides for the method as disclosed herein wherein the small molecule is amitriptyline, imipramine, LM 22A4 (N,N′,N″Tris(2-hydroxyethyl)-1,3,5-benzenetricarboxamide), 7,8-dihydroxyflavone (DHF), 7,8,3′-Trihydroxyflavone (THF), Mab2256, neurotrophin-4 (NT-4), neurotrophin-3 (NT-3), brain derived neurotrophic factor (BDNF), nerve growth factor (NGF), N-acetylserotonin, N-[2-(5-Hydroxy-1H-indol-3-yl)ethyl]-2-oxo-3-piperidinecarboxamide (HIOC), deoxygedunin, LM-22A4, or tricyclic dimeric peptide 6 (TDP6)).

In some embodiments of all aspects, the small molecule is administered up to 12, 10, 8, 6, 4, 2, or one hour before, or 1-12, 2-12, 2-6, 6-12, or 2-8 hours before, initiation of the noise exposure. In some embodiments of all aspects, the small molecule is administered within 24 hours, 12 hours, 10 hours, 8 hours, 6 hours, 4 hours, 2 hours or one hour, e.g., 0-2, 0-4, 0-6, 0-8, 0-10, 0-12, 0-18, or 0-24 hours after termination of the noise.

In some aspects, this disclosure provides for a method of treating or reducing the risk of hidden hearing loss (HHL) in a subject, the method comprising administering to the subject a therapeutically effective amount of a small molecule therapeutic Trk agonist, e.g., a TrkB and/or TrkC agonist.

In some aspects, this disclosure provides for the use of a small molecule Trk agonist, e.g., a TrkB and/or TrkC agonist, for treating or reducing the risk of developing hidden hearing loss (HHL) in a subject.

In some embodiments of all aspects of the methods described herein, the small molecule is amitriptyline, imipramine, LM 22A4 (N,N′,N″Tris(2-hydroxyethyl)-1,3,5-benzenetricarboxamide), 7, 8-dihydroxyflavone (DHF), 7,8,3′-Trihydroxyflavone (THF), Mab2256, neurotrophin-4 (NT-4), neurotrophin-3 (NT-3), brain derived neurotrophic factor (BDNF), nerve growth factor (NGF), N-acetylserotonin, N-[2-(5-Hydroxy-1H-indol-3-yl)ethyl]-2-oxo-3-piperidinecarboxamide (HIOC), deoxygedunin, LM-22A4, or tricyclic dimeric peptide 6 (TDP6)).

In some embodiments of all aspects, the small molecule is administered orally or locally to the ear of the subject.

In some embodiments of all aspects, the method comprises identifying and/or selecting a subject who has hidden hearing loss. In some embodiments of all aspects, the method of identifying and/or selecting a subject who has hidden hearing loss comprises: measuring a neural-based auditory evoked potential (e.g., auditory brainstem response (ABR) or compound action potential (CAP) in a subject); measuring hair-cell-based responses (e.g. distortion product otoacoustic emissions (DPOAE), summating potentials (SP), or SP/AP ratio in the subject; and identifying a subject who has a reduced Wave I on ABR or CAP as compared to a normal-hearing subject, and a normal DPOAE, SP, or SP/AP ratio, as having HHL.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-B are graphs showing synapse loss for two common causes of human hearing loss; noise and aging. FIG. 1A shows that IHC synapses are lost acutely and permanently after noise exposure (FIG. 1A). In unexposed animals, synapse loss is gradual, throughout the lifespan and throughout the cochlea (FIG. 1B). Synapses were quantified as juxtaposed pairs of presynaptic ribbons and postsynaptic glutamate receptors in unexposed and exposed (8-16 kHz, 100 dB SPL, 2 h at 16 wk) male CBA/CaJ mice, across a broad range of log-spaced cochlear frequencies. For B, means (±SE) are normalized to 4 wk values. Data from: Kujawa and Liberman (2009) (1A) and Sergeyenko et al. (2013) (1B).

FIGS. 2A-C are graphs showing that permanent reductions in neural-based auditory brainstem response (ABR Wave 1), but not outer hair cell-based distortion product otoacoustic emissions (DPOAE) amplitudes, are seen in ears with recovered thresholds after noise. Shown are DPOAE (2A) and ABR Wave I (2B) response growth functions in the region of maximum acute noise-induced threshold shift, 1 d and 8 wk after exposure to 16 wk male CBA/CaJ; unexposed controls shown for comparison. This ˜50% neural response decrement at 8 wk (FIG. 2B) was associated with ˜50% loss of synapses (FIG. 1A). Similarly, neural response amplitude declines are proportional to synaptic and neural losses in aging (to 128 wks) CBA/CaJ, where synapse survival at several cochlear locations (re: values at 4 wk) is plotted as a function of age (FIG. 1B) and vs mean Wave I amplitudes at 80 dB SPL (FIG. 2C). Panels A, B modified from Kujawa and Liberman (2009); Panel C from Sergeyenko et al. (2013).

FIGS. 3A-D show that imipramine promoted spiral ganglion neurite growth in a dose-dependent manner. Spiral ganglion neurons were isolated from P4 CBA/CaJ mouse cochlea and cultured with DMEM/F12 supplemented with N2 and B27 in a 37° C. incubator with 5% CO2 (control conditions)(FIG. 3A). For drug treatment, BDNF (50 ng/ml) (FIG. 3C) or imipramine (1 uM or 5 uM)(FIG. 3B or FIG. 3D) was added. Two days after culture, explants were immunostained with a neurofilament antibody. Shown for comparison, BDNF treatment promoted neurite outgrowth, as compared to control conditions.

FIGS. 4A-B show amitriptyline induced cochlear afferent synaptogenesis. Isolated SGNs and denervated organ of Corti were extracted from P4/5 CBA/CaJ cochlea, the co-cultured for 6 days (see Brugeaud et al. (2014) Dev Neurobiol. April; 74(4):457-66 and Tong et al. (2013) J Assoc Res Otolaryngol. 14(3):321-9 for detailed methods). In some co-cultured explants, amitriptyline (AT; 0.5 uM) was added (4A, 4B). After culture, explants were fixed and immunostained with antibodies against neurofilament (green), CtBP2 (blue) and PSD-95 (red). Confocal images were taken in the IHC region. Newly generated afferent synapses were identified by juxtaposed CtBP2/PSD-95 puncta (yellow arrows). Synapse counts were greater in AT-treated than in untreated controls.

FIGS. 5A-D show synapses (5A and 5B) and response growth functions (5C and 5D) of amitriptyline (AT) treated ears in vivo. AT-treated ears demonstrated more synapses than saline treated controls at short post-exposure times (5A), and persisting to at least one year after synaptopathic exposure (5B); effects were dose-responsive. Correspondingly, ABR Wave 1 amplitudes (5D), but not DPOAE amplitudes (5C) were larger in AT-treated ears at 52 wk.

FIG. 6 shows the effects of amitriptyline (AT)-treated ears in vivo with a single dose of drug.

### DETAILED DESCRIPTION

Noise exposure can produce temporary and permanent changes in threshold sensitivity. Permanent threshold losses after noise are associated with permanent cochlear injury, often hair cell loss or damage. In contrast, complete post-exposure recovery of thresholds has been assumed to indicate recovery from underlying cochlear injury and no persistent or delayed consequences for auditory function as noise-exposed individuals age (Noise and Military Service: Implications for Hearing Loss and Tinnitus (2006). L E Humes, L M Joellenbeck, J S Durch (eds). The National Academies Press, Wash. D.C.). These assumptions form the basis for noise exposure guidelines, they shape assessments of noise-induced injury in the laboratory and in the clinic and they guide approaches to treatment and prevention.

Recent work in mouse models of noise and aging, however, challenges this view and reveals what is likely a very common consequence of noise exposure (Kujawa et al. (2006). J. Neurosci. 26(7):2115-2123; Kujawa et al. (2009); Sergeyenko et al. (2013); Fernandez et al. (2015)). Noise produces immediate and widespread loss of synapses between inner hair cells (IHC) and cochlear neurons and, with a delay, loss of the affected neurons themselves. This loss of communication changes the way acoustic information is processed by the ear, even when thresholds recover and no hair cell loss will ensue. Further, the noise-induced insult can progress, long after the noise has stopped, accelerating changes that otherwise occur with age and expanding to involve cochlear regions remote from the initial insult.

Reduced neural output from the cochlea may be a significant precipitating event in the generation of tinnitus after noise exposure (Roberts et al. (2010). J Neurosci 30(45):14972-14979). The discovery of noise-induced synaptopathy/primary neuropathy has inspired studies linking tinnitus with greater loss of cochlear synapses and ABR Wave I amplitudes in an animal model (Ruttiger et al. (2013). PLoS One 8(3):e57247) and with reduced ABR wave 1 in patients with normal audiograms (Gu et al. (2010). J Neurophysiol. 104(6):3361-3370; Schaette and McAlpine (2011). J Neurosci. 31(38):13452-13457). Using acoustic startle in mice, noise-induced primary neuropathy has been linked with hyperacusis, a phenomenon often associated with tinnitus (Hickox and Liberman (2014). J Neurophysiol. 111(3):552-564). Thus, the present methods can also be used to treat tinnitus.

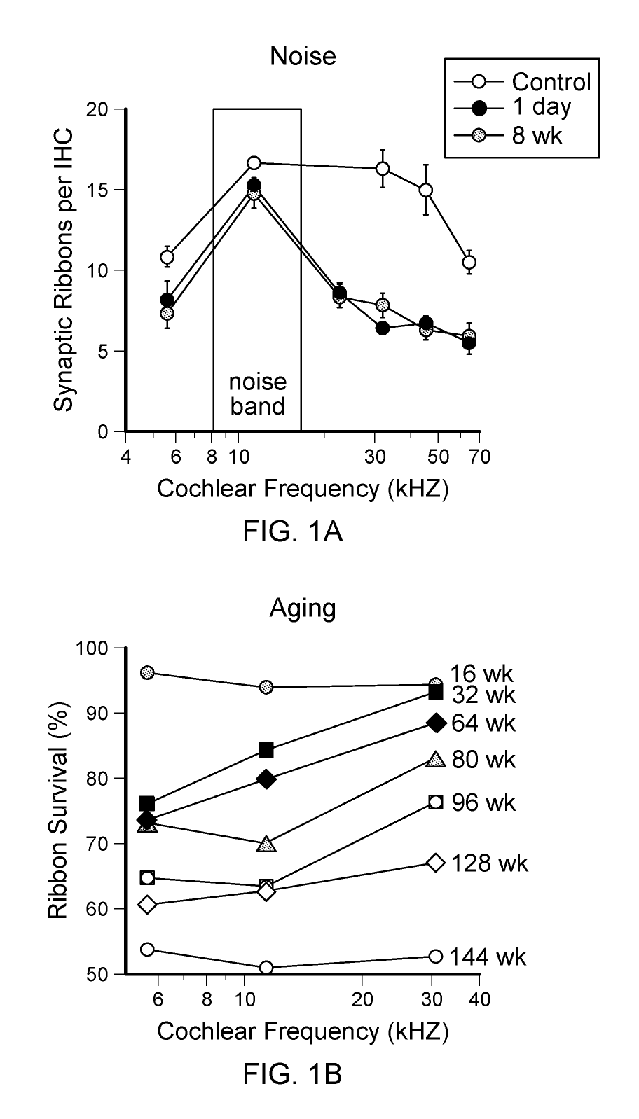

Age-related loss of IHC-cochlear nerve synapses may be an early contributor to the performance declines of aging listeners. In ears that age normally, e.g., without noise exposure, there is gradual loss of cochlear nerve synapses, as shown in FIG. 1B. Published work (Sergeyenko et al. (2013) and Fernandez et al. (2015)) shows that, by the end of the CBA/CaJ mouse's lifespan, roughly 40% loss is evident, throughout the cochlea. Cochlear nerve cell bodies (spiral ganglion cells, SGC) show proportional declines, and these losses in aging CBA/CaJ are consistent with our findings in an age-graded series of human temporal bones with full complements of hair cells (Makary et al. (2011). J Assoc Res Otolaryngol. 12(6):711-717).

Noise produces similar synaptic losses, but immediately, and then accelerates aging. After noise, up to ˜40% loss can be seen by 1 hr post exposure (FIG. 1A). Losses at short post-exposure times are restricted to cochlear regions with maximum acute threshold shift, e.g. ˜32 kHz, for this exposure (Kujawa et al. (2009) and Fernandez et al. (2015)). Ganglion cell losses follow with a delay, first in regions of maximum acute threshold shift. As animals age, synapse and proportional SGC losses expand to cochlear regions that initially appear uninvolved in the noise insult (Fernandez et al. (2015)). These effects of noise are seen whether the exposure produced a robust temporary threshold shift (TTS) only (Kujawa et al. (2009) and Fernandez et al. (2015)), or resulted in permanent threshold shift (PTS) (Kujawa et al. (2006)).

Prior work on noise-induced hearing loss concentrated on threshold shift, which is primarily a measure of hair cell damage. For high-level noise, hair cell loss to can be seen in minutes to hours, whereas ganglion cell loss is not seen for weeks to months (Spoendlin (1971). Acta Otolaryng 71:166-176; Johnsson (1974). Ann Otol Rhinol Laryngol 83:294-303; Lawner et al. (1997). Int J Dev Neurosci 15:601-617). This difference in degenerative time course, and the correlation, in long-surviving ears, between regions of hair cell loss (particularly IHCs) and regions of SGC death (Liberman and Kiang (1978). Acta Otolaryngol Suppl 358:1-63), has suggested that hair cells are the primary targets of acoustic overexposure, whereas noise-induced SGC death occurs only as a secondary event to the loss of hair cells and, perhaps, of the neurotrophins they provide (Glueckert et al. (2008). J Comp Neurol 507:1602-1621). In contrast, the present inventors' recent work shows that noise-induced SGC death can be extensive despite a normal hair cell complement (Kujawa et al. (2006), Kujawa et al. (2009), Lin et al. (2011), Fernandez et al. (2015)).

This neurodegenerative consequence of noise has remained hidden for many years. The initial event, the loss of IHC synaptic ribbons and unmyelinated synaptic terminals contacting IHCs, is not visible in routine light microscopy without special immunostaining (see Methods). Long-delayed loss of the neuronal cell bodies, which is easily seen in the light microscope, was not linked to noise when no noise-induced hair cell loss occurred. Additionally, it remained undetected because changes in function after noise are typically assessed using thresholds and, although thresholds are sensitive metrics of hair cell damage, they are relatively insensitive to diffuse loss of cochlear synapses and cochlear neurons (Kujawa et al. (2006); Kujawa et al. (2009); Sergeyenko et al. (2013); Fernandez et al. (2015); Schuknecht and Woellner (1953). Laryngoscope 63:441-465; Liberman et al. (1997). Auditory Neuroscience 3:255-268; El-Badry and McFadden (2007). Brain Res 1134:122-130; Earl and Chertoff (2010). Ear Hear. 31(1):7-21); Lobarinas et al. (2013). Hear Res. 2013 August; 302:113-20): a) DPOAEs are unaffected because only pre-synaptic processes are required for their generation; b) neural response thresholds (CAP, ABR) are unaffected, because the noise targets cochlear neurons with high thresholds, as discussed below (Furman et al. (2013). J Neurophysiol. 110(3):577-586) and c) behavioral audiometric thresholds are unaffected, for the same reason as (b) and because stimulus detection requires less neural information than stimulus discrimination. Thus, this condition has become known as “hidden hearing loss.”

The identification of previously unrecognized acute and long-term consequences of noise exposure has important noise-risk implications for anyone who is chronically or acutely exposed to noise. Whereas, therapeutic efforts that aim to protect hair cells and thresholds from noise-induced declines are important, they appear to target events occurring rather late in the degenerative processes that we have characterized. As shown herein, cochlear synapse and ganglion cell preservation can be achieved with small molecule therapeutics.

Methods of Treating or Reducing Risk of Developing Hidden Hearing Loss

Described herein are methods for treating, or reducing the risk of developing, hidden hearing loss, using small molecule Trk (A, B or C) agonists, e.g., amitriptyline, imipramine, LM 22A4 (N,N′,N″Tris(2-hydroxyethyl)-1,3,5-benzenetricarboxamide), 7, 8-dihydroxyflavone (DHF), 7,8,3′-Trihydroxyflavone (THF), Mab2256, neurotrophin-4 (NT-4), neurotrophin-3 (NT-3), brain derived neurotrophic factor (BDNF), nerve growth factor (NGF), N-acetylserotonin, N-[2-(5-Hydroxy-1H-indol-3-yl)ethyl]-2-oxo-3-piperidinecarboxamide (HIOC), deoxygedunin, LM-22A4, or tricyclic dimeric peptide 6 (TDP6) as an active ingredient). These agents have been reported to mimic the neuroprotective effects of neurotrophins by TrkB receptor agonist, but with demonstrated better bioavailability when delivered to the systemic circulation (Jang et al. (2009). Chem Biol 16(6):644-656; Jang et al. (2010). Proc Natl Acad Sci USA 107(6):2687-2692; Yu et al. (2013). J Neurosci. 33(32):13042-13052).

A subject who has hidden hearing loss (HHL) can be identified by reductions in the neural Wave 1 (measured by auditory brainstem response (ABR) or compound action potential (CAP)), preferably in the absence of distortion product otoacoustic emissions (DPOAE) changes (and preferably in the absence of changes in Summating potential (SP) or SP/Action Potential (SP/AP) ratio, see Sergeyenko et al., 2013), at least until OHC loss begins; this indicates dysfunction in IHCs, cochlear neurons, or the synaptic transmission between them (see, e.g., Starr et al. (2008) “Perspectives on Auditory Neuropathy: Disorders of Inner Hair Cell, Auditory Nerve, and Their Synapse.” In: Basbaum et al., editors. The Senses: A Comprehensive Reference, Vol 3, Audition, Peter Dallos and Donata Oertel. San Diego: Academic Press, p. 397-412). In animal models, aging and noise-exposed ears without hair cell loss show proportional loss of synapses and Wave I amplitude, up to about 50% loss. Synaptopathy is also present in ears with elevated thresholds, e.g., from noise, drugs, or aging, in which case the DPOAEs will not be normal. These individuals would also be candidates for treatment. Subjects with demonstrated HHL can be treated using the methods described herein, by administration of a small molecule BDNF- or NT-3-mimicking TrkB or TrkC agonists, e.g., amitriptyline, imipramine, LM 22A4, DHF, THF, Mab2256, neurotrophin-4 (NT-4), neurotrophin-3 (NT-3), brain derived neurotrophic factor (BDNF), nerve growth factor (NGF), N-acetylserotonin, N-[2-(5-Hydroxy-1H-indol-3-yl)ethyl]-2-oxo-3-piperidinecarboxamide (HIOC), deoxygedunin, LM-22A4, tricyclic dimeric peptide 6 (TDP6) or TrkA/B/C agonists as an active ingredient.

In the present methods, a subject who is at risk for developing hidden hearing loss is one who will be or is over-exposed to sound (noise) or certain ototoxic drugs, e.g., a permanent threshold shift (PTS)- or temporary threshold shift (TTS)-inducing level of exposure, e.g., someone who is occupationally or recreationally exposed to noise, or who receives a synaptopathy-producing ototoxic drug (Liu et al. 2013) as part of a medical therapy, or who is intending to be exposed to noise, e.g., at a concert or construction site. Individuals also develop hidden hearing loss as they age; speech-in-noise and temporal processing difficulties are well known to be well underway by middle age (Abel et al., (1990). Scand Audiol 19:43-54; Grose et al. (2006). J Acoust SocAm119:2305-2315; Grose and Mamo (2010). Ear Hear 31:755-760); Snell and Frisina, (2000). J Acoust Soc Am 107:1615-1626), and are progressive, long before the audibility declines that arise from threshold elevations and hair cell loss (Sergeyenko et al. (2013)). These subjects can be treated using the methods described herein, by administration of a small molecule TrkB,C agonist, e.g., amitriptyline, imipramine DHF, or THF, e.g., by administration of one dose up to 12 hours before an episode of noise exposure, e.g., up to 12, 10, 8, 6, 4, 2, or one hour before, or 0-12, 0-6, 1-12, 2-12, 2-6, 6-12, or 2-8 hours before, initiation of the noise exposure, and/or optionally one or more doses after the end of the episode of noise exposure, e.g., at least one dose within 0 to 12 or 24 hours after termination of the noise, e.g., within 24 hours, 12 hours, 10 hours, 8 hours, 6 hours, 4 hours, 2 hours, one hour or immediately after noise (0 hours), e.g., 0-2, 0-4, 0-6, 0-8, 0-10, 0-12, 0-18, or 0-24 hours after termination of the noise. In some embodiments, the subject does not yet have HHL (e.g., has normal Wave I/normal ABR/CAP, and normal DPOAE).

Generally, the methods include administering a therapeutically effective amount of a small molecule as described herein to a subject who is in need of, or who has been determined to be in need of, such treatment.

As used in this context, to “treat” means to ameliorate at least one symptom of HHL, e.g., speech-in-noise difficulties, and other abnormal auditory perceptual phenomena like tinnitus, that occur in noise-exposed individuals, with or without threshold sensitivity loss. Administration of a therapeutically effective amount of a compound described herein for the treatment of HHL may result in a reduction in tinnitus perception and a return or approach to normal sound perception. In these subjects, regrowth of neurites and synapses may result in these improvements in hearing.

As used herein, “reducing the risk” of developing hidden hearing loss means to reduce the risk that a subject who is aging and/or is exposed to noise or an ototoxic drug, e.g., a PTS- or TTS-inducing insult, will later develop HHL (without wishing to be bound by theory or mechanism, this is believed to be the result of loss of synapses or neurons); their risk is reduced as compared to someone who does not receive treatment using methods described herein, and who is aging or is exposed to the same noise or ototoxic agent, e.g., PTS- or TTS-inducing noise or drug.

Pharmaceutical Compositions and Methods of Administration

The methods described herein include the use of pharmaceutical compositions comprising small molecule Trk agonists, e.g., TrkB agonists (e.g., amitriptyline; imipramine; LM 22A4 (N,N′,N″ Tris(2-hydroxyethyl)-1,3,5-benzenetricarboxamide); 7,8-dihydroxyflavone (DHF); 7,8,3′-Trihydroxyflavone (THF); neurotrophin-4 (NT-4); neurotrophin-3 (NT-3); brain derived neurotrophic factor (BDNF); nerve growth factor (NGF); N-acetylserotonin; N-[2-(5-Hydroxy-1H-indol-3-yl)ethyl]-2-oxo-3-piperidinecarboxamide (HIOC); deoxygedunin; LM-22A4; or tricyclic dimeric peptide 6 (TDP6); TrkC agonists (e.g., Mab2256) or TrkA/B/C agonists as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, or subcutaneous; oral; nasal (e.g., inhalation); transdermal (topical); or rectal administration. In some embodiments, oral administration is preferred.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

Detailed Methodology

The following materials and methods were used in the examples set forth herein to characterize the synaptopathy/neuropathy (hidden hearing loss) and to document the protective/regenerative effects of Trk agonists in vitro and in vivo.

Animals: Experiments were carried out in CBA/CaJ mice. CBA/CaJ is a useful reference for studies of both noise-induced and age-related hearing loss and to cochlear injury. It shows noise vulnerability similar to that observed in other small experimental mammals except, of course, certain ‘resistant’ or ‘vulnerable’ inbred mouse strains (Li (1992). Acta Otolaryngol. 112:956-967; Erway et al. (1996). Hear Res. 93:181-187; Yoshida et al. (2000). Hear Res. 141:97-106; Candreia et al. (2004). Hear Res. 194:109-117); Street et al. (2014). J Assoc Res Otolaryngol. 2014 October; 15(5):721-38). With respect to aging, after normalizing for cochlear place and relative lifespan, the pace of age-related threshold elevation and SGN loss in CBA/CaJ looks remarkably like that seen in human data (Sergeyenko et al. (2013)). Thus, the strain is particularly appropriate to provide the optimization of bioassays and treatments for application to the human.

All mice were born and reared in our animal care facility (inbred breeders replaced every three generations to maintain isogeneity with supplier stocks) and held to various ages as described. The acoustic environment of this facility has been characterized by both spectral analysis and longitudinal noise-level data logging (Sergeyenko et al. (2013)).

In Vivo Studies.

Noise Exposure: Noise exposure stimuli were generated by a waveform generator (Tucker-Davis WG1), bandpass filtered with >60 dB/octave slope (Frequency Devices), amplified (Crown D-75) and delivered (JBL compression driver) through an exponential horn extending into a small, reverberant exposure chamber. Exposures were delivered to awake animals held within small cells of a subdivided cage, one animal/cell, suspended directly below the horn of the sound-delivery loudspeaker. Noise calibration to target SPL was performed immediately before each exposure session. Sound pressure levels varied by <1 dB across all cells (Kujawa et al. (2006); Kujawa et al. (2009)).

Cochlear Function: Detailed techniques have been described in previous publications (Kujawa et al. (2006); Kujawa et al. (2009); Sergeyenko et al. (2013)). All acoustic stimuli were digitally generated. All physiologic tests were conducted in an acoustically and electrically shielded and heated chamber, using a National Instruments PXI-based system and 24-bit I/O boards controlled with custom LabVIEW software. The custom acoustic system comprised two miniature dynamic earphones as sound sources (CDMG15008-03A; CUI) and an electret condenser microphone (FG-23329-PO7; Knowles) coupled to a probe tube to measure sound pressure near the eardrum.

Effects of noise on the cochlea and its interaction with age were assessed physiologically using outer hair cell-based distortion product otoacoustic emissions (DPOAEs) and neural-based auditory brainstem responses (ABRs, specifically, wave 1). Response thresholds and suprathreshold response growth functions were recorded in ketamine/xylazine-anesthetized mice. DPOAEs were measured in response to f1 and f2 primaries (f2/f1=1.2, f2=ABR test frequencies; L2=L1−10 dB). The DPOAE at 2f1−f2 (with surrounding noise floor) was extracted from the ear canal sound pressure and threshold was computed by interpolation as the primary level (f2) required to produce a DPOAE of −5 dB SPL. ABRs were recorded with 5 ms tone-pips (0.5 msec rise/fall, alternating polarity) for the same range of frequencies. Wave 1 thresholds and wave peak ratios were determined by custom offline analysis routines.

Cochlear Tissue Processing and Analysis: Techniques for cochlear fixation, dissecting and immunostaining are described in prior publications, as are techniques for quantification of pre- and post-synaptic structures in highpower confocal z-stacks (Kujawa et al. (2009); Sergeyenko et al. (2013)). Briefly, deeply anesthetized mice were intracardially perfused, both cochleae were extracted, and the round and oval windows opened to allow intra-labyrinthine perfusion of the same fixative. One cochlea was processed for immunostained cochlear whole mounts and the other for plastic embedding. We triple stained for pre-synaptic ribbons (CtBP2), post-synaptic glutamate receptors (GluA2) and/or cochlear nerve terminals (Na+K+ATPase); myosin VIIA aided hair cell visualization. For cochlear whole mounts, analysis began with measurement of the frequency map for each dissected whole mount, using low-power images of each immunostained piece and a custom plug-in to Image J that computes and displays the location of half-octave frequency points using published distance to frequency algorithms for the mouse (Muller et al. (2005). Hear Res 202:63-73). High-power, confocal image stacks were obtained at evenly spaced locations along the cochlear spiral, including regions of lesion focus. Given the stereotyped sectioning angle, these locations correspond roughly to the 6, 12, 22, 32, 45 and 64 kHz regions. At each locus, high-NA (1.3) objectives were used to obtain a complete confocal z-stack through the synaptic zones of all IHCs and OHCs. In each field, hair cells were counted under DIC optics, and expressed pre- and post-synaptic elements on a per hair cell basis. For afferent innervation, synaptic ribbons were counted in IHC areas, and percentages of ribbons with closely apposing glutamate receptor patches or terminals assessed, aided by the use of Amira software to enable a true 3-D analysis of the volumes of immunostained structures, and custom software to isolate the voxel space around each structure of interest.

For quantification of spiral ganglion cells, cochleae were additionally postfixed, decalcified and osmicated and embedded in EPON in a stereotyped orientation. Plastic-embedded, osmium-stained sections (10 μm) were cut in a roughly horizontal plane parallel to the modiolus. In each case, ganglion cell counts were made in the section precisely in the middle of each half turn, where the cutting angle is perpendicular to the spiral of Rosenthal's canal, at regions corresponding to the synapse counts. In each selected section, the area of Rosenthal's canal is traced, the number of ganglion cells within was counted, including only those cells with a visible nucleus and nucleolus, and the cell density is computed (cells/10,000 μm²). All morphological analyses were performed by an individual having no knowledge of animal age or treatment, with subsets double counted by a second individual as a check for consistency. For both the physiologic and histologic assessments, post-exposure survivals ranged from 0 h to 52 wk, to capture the magnitude of the initial insult as well as to assess recovery/progression of the pathology and the early and late effects of test compounds. Unexposed, as well as unexposed, saline-treated animals, held identically except for treatments, served as controls.

### Example 1: Cochlear Function: Neural Response Amplitudes Reveal What Thresholds do not

In studying functional consequences of such loss, we have employed two complementary techniques. The auditory brainstem response (ABR) and the compound action potential (CAP), measured from scalp or round-window electrodes respectively, are sound-evoked potentials generated by neuronal circuits in the ascending auditory pathways: the first ABR or CAP wave represents summed activity of the cochlear nerve (Buchwald and Huang (1975). Science 189:382-384; Antoli-Candela F, Jr., Kiang N Y S (1978). “Unit activity underlying the N1 potential.” In: Evoked Electrical Activity in the Auditory Nervous System (Naunton R F, Fernandez C, eds), pp 165-191. New York: Academic Press). To complement these measures of cochlear output, we assess OHC function via distortion product otoacoustic emissions (DPOAEs). These acoustic signals are created and amplified by the cochlear epithelium and measured in the ear canal. They require the biological motors in OHCs (Liberman et al. (2002). Nature 419(6904):300-304), which amplify sound-evoked cochlear vibration. They do not require IHCs or auditory nerve fibers for their generation (Kujawa et al. (2009) and Takeno et al. (1994). Hear Res 75:93-102). Thus, reductions in the neural Wave 1, in the absence of DPOAE changes, indicate dysfunction in IHCs, cochlear neurons, or the synaptic transmission between them.

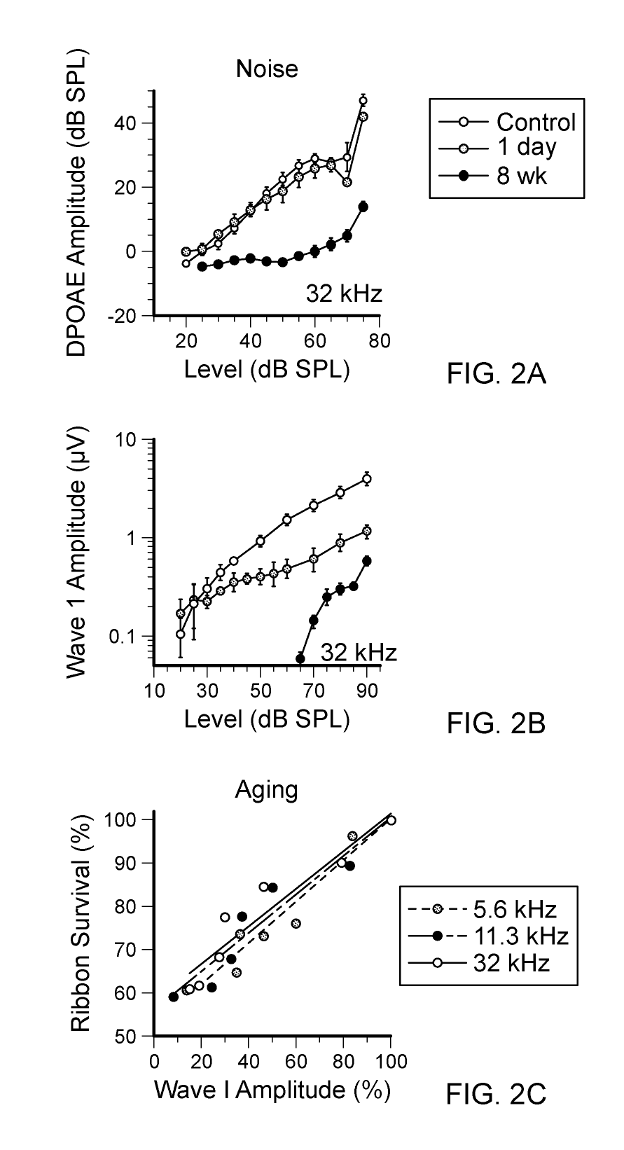

Hallmark findings in ears receiving this synaptopathic, TTS-producing exposure include a permanent decrease in Wave I amplitude in ears with complete recovery of DPOAE and Wave I thresholds and with restored DPOAE amplitudes, shown, for example, in FIG. 2A vs. 2B (Kujawa et al. (2009) and Fernandez et al. (2015)). Neural amplitude declines are sensitive, in magnitude and cochlear frequency, to the underlying synaptopathy and the delayed cochlear nerve loss. Similarly, in the case of aging, neural-based Wave I amplitudes fall at a faster pace than seen in the OHC-based DPOAEs (Sergeyenko et al. (2013)). Here, too, declines are proportional to the synaptopathy, as shown in FIG. 2C, at least until OHC loss commences (Sergeyenko et al. (2013) and Fernandez et al. (2015)). This close correspondence between synapse survival and Wave 1 amplitude in both aging and after TTS-producing noise, suggests that this non-invasive auditory test could be useful in diagnosing cochlear synaptopathy in humans, at least among those with near-normal threshold audiograms.

Is it paradoxical that thresholds return to normal despite this dramatic loss of the nerve fibers connecting hair cells to the brain? Neurophysiological study in our laboratories (Furman et al. (2013). J Neurophysiol. 110(3):577-586) suggests that neural loss after TTS is selective for the subset of cochlear nerve fibers comprising ˜40% of the population, with high thresholds and low spontaneous firing rates (low-SR) (Liberman (1978). J Acoust Soc Am. 63(2): p. 442-455; Winter et al. (1990). Hear Res 45: 191-202). Selective low-SR neuropathy in our aging and noise-exposed mice would explain why a substantial loss of IHC-afferent fiber synapses (FIGS. 1A-B) has minimal effect on neural response thresholds, but is proportionately reflected in neural response amplitude declines (FIGS. 2A-C).

In normal ears, low-SR fibers are less susceptible to continuous noise masking (Costalupes et al. (1984). J Neurophysiol 51:1326-1344); moderate-level noise that completely masks sound-evoked rate-responses in high-SR fibers can leave low-SR fibers unaffected, by virtue of their higher thresholds. This has led to the view that we hear with our high-SR fibers in quiet, and with our low-SR fibers in a noisy background (Costalupes et al. (1984). J Neurophysiol 51:1326-1344). Difficulty hearing in noise is a classic complaint in many forms of sensorineural involvement and as individuals age, even when thresholds are well preserved (Costalupes et al. (1984). J Neurophysiol 51:1326-1344; Gordon-Salant (2005). J Rehabil Res Dev 42 [Suppl 2]:9-24). Loss of low-SR fibers in noise-exposed, aging ears may thus be an important contributor to declining auditory performance, particularly with respect to speech-in-noise difficulties. Low-SR neurons recover more slowly than high-SR neurons from prior stimulation (Relkin and Doucet (1991). Hear Res. 55(2):215-222).

### Example 2. The Role of Neurotrophic Support in Treatment

The noise-induced damage to cochlear nerve terminals, and the subsequent loss of the neurons themselves, may arise directly from an acute, ‘excitotoxic’ event instigated by the noise. Swelling of cochlear nerve terminals is seen in the IHC area minutes to hours after overexposure, even when threshold shifts are ultimately reversible (Robertson (1983). Hear Res. 9:263-278). It is present after the synaptopathic exposure we have described here. The same acute swelling is observed after cochlear perfusion of glutamate agonists, and is minimized by glutamate antagonists, with the same recovery of cochlear neural thresholds (Puel et al. (1991). Neurosci. 45(1):63-72; Puel et al. (1994). J Comp Neurol. 341:241-256). We have hypothesized (Kujawa et al. (2009)) that the afferent terminal retraction that follows the acute excitotoxic swelling interrupts necessary neurotrophic support, ultimately resulting in the death of affected neurons

Neurotrophins, e.g., BDNF and NT-3, are necessary for the survival of spiral ganglion neurons (Fritzsch et al. (2004). Prog Brain Res. 146:265-278). Some neurotrophins, and drugs that act like neurotrophins at the same Trk receptors, have demonstrated neuroprotective effects after kainate-induced neuro-excitotoxic insult in the hippocampus (Jang et al. (2009). Chem Biol 16(6):644-656; Jang et al. (2010). Proc Natl Acad Sci USA 107(6):2687-2692). The latter small molecule therapeutics offer improved bioavailability in vivo. Thus, as an indirect test of the hypothesis, we conducted preliminary experiments in vivo and in vitro to determine whether such drugs (amitriptyline, imipramine) can influence auditory nerve function and survival (Lall et al. (2013). Neurotrophin-rescue of spiral ganglion neurons after noise. ARO MidWinter Meeting, 2013, Abstract; Tong et al. (2014). Increased survival of spiral ganglion neurons in auditory neuropathy by treatment with small molecule Trk receptor agonists. ARO MidWinter Meeting, 2014, Abstract).

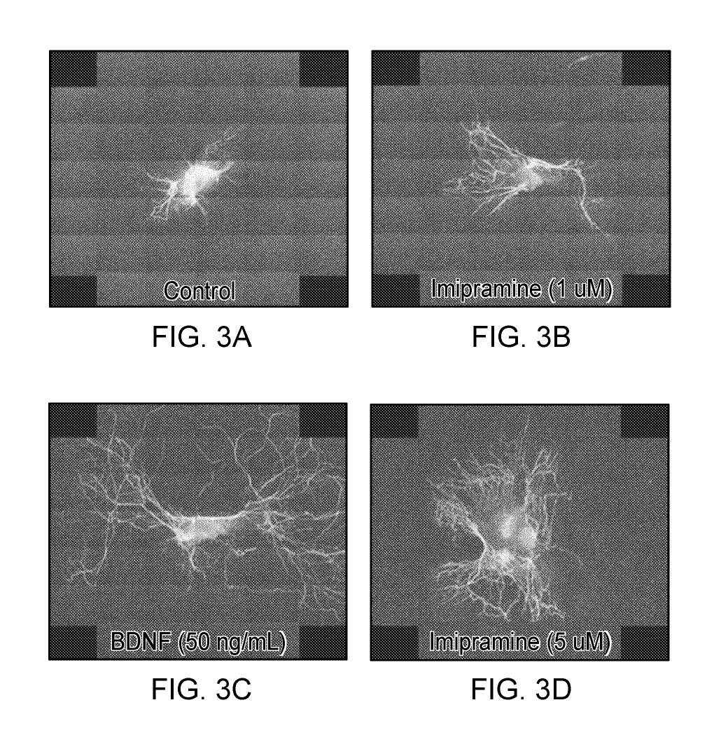

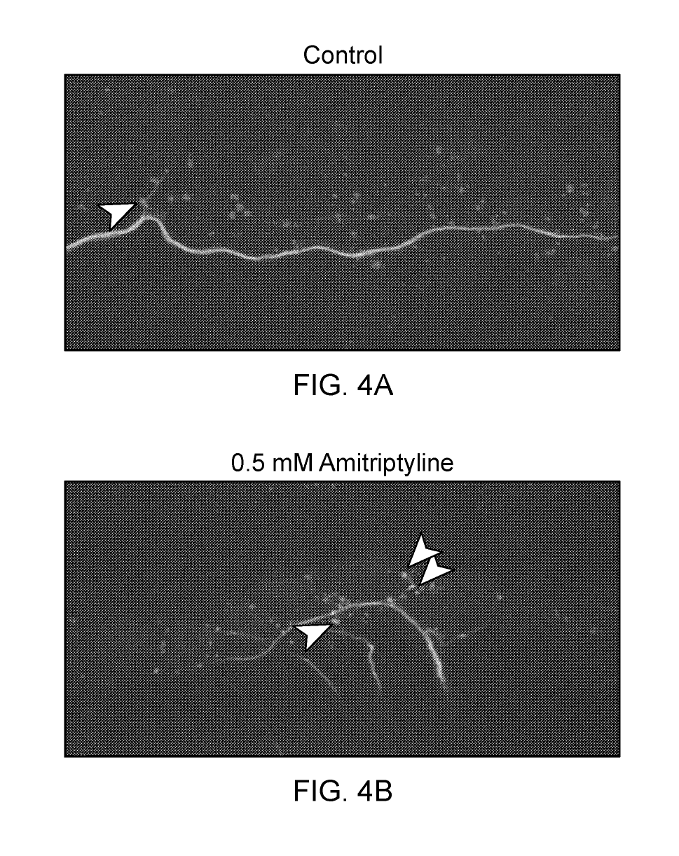

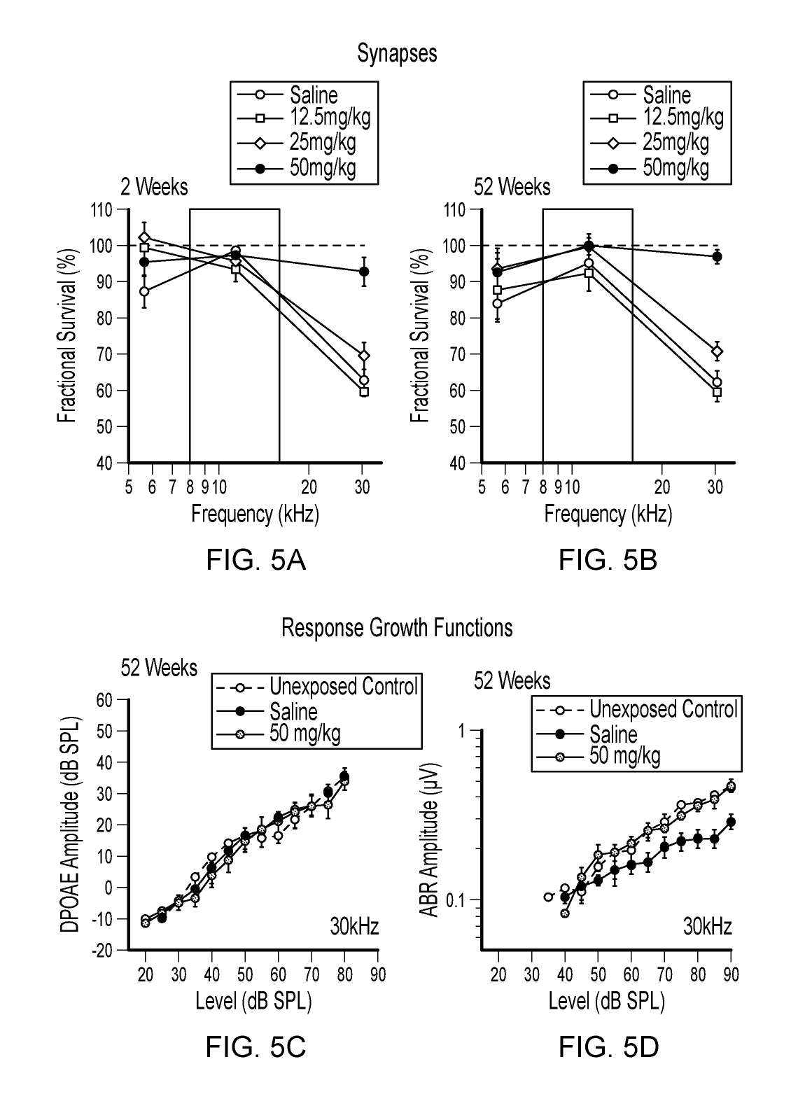

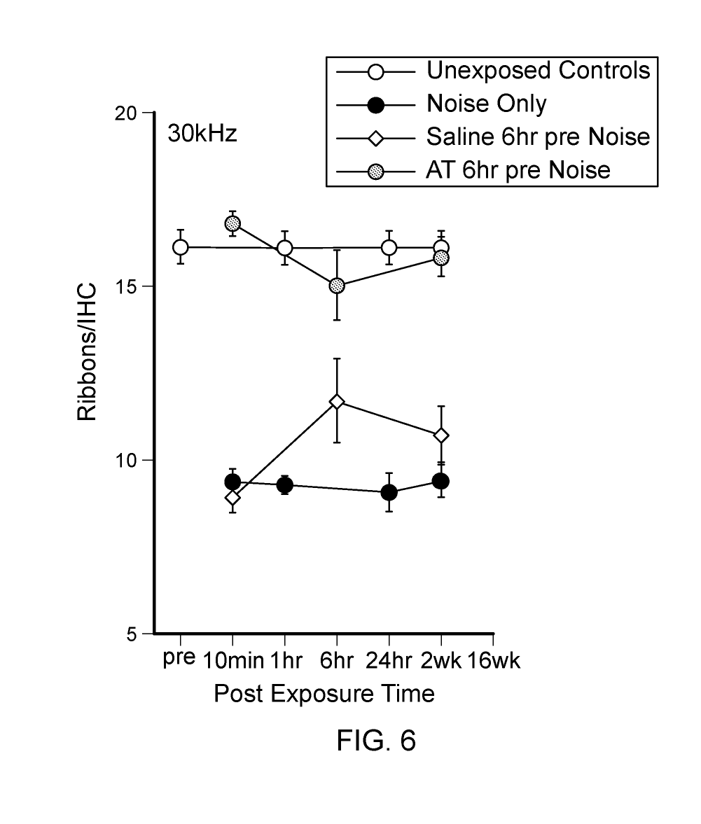

In vitro, we observed dose-responsive dendritic sprouting (imipramine; FIG. 3) and synaptogenesis (amitriptyline; FIGS. 4A-4B). In vivo, one year post excitotoxic noise, neural response amplitudes were larger and cochlear synaptic and ganglion cell counts were greater in amitriptyline-treated animals relative to saline-treated controls (FIGS. 5A-D). These effects were dose-responsive and could be seen even with a single dose of drug (amitriptyline, FIG. 6). Compared to the short-lived benefit of exogenously-applied neurotrophins, which decline rapidly once delivery stops (Gillespie and Shepherd (2005). Eur J Neurosci. 22(9):2123-2133), initial results showed that amitriptyline had a long period of post-drug efficacy.

### Example 3. Protection of Synapses Between Hair Cells and Spiral Ganglion Neurons

In Vitro Studies.

Afferent synapses can be ablated by kainate administration, which mimics the effects of noise damage to peripheral axons of SGN, including retraction of the peripheral fibers. When performed in a newborn organ of Corti in vitro, the axons regenerate to contact hair cells and make new synapses. This system is used to further test Trk agonists for effects on the loss of peripheral synapses (Tong et al. (2013). J Assoc Res Otolaryngol. 14(3):321-329).

The organ of Corti is isolated to perform explant experiments. The cochlea is dissected from 3 to 5 day old CBA/CaJ mice. The heads are bisected midsagittally, the cochleas removed and dissected in ice cold Hank's balanced salt solution (HBSS), gently freeing the otic capsule and spiral ligament. The tissue is oriented in a 4-well dish coated with fibronectin so that the apical surfaces of the hair cells are pointing up and the basilar membrane is directed toward the fibronectin substrate. Excitotoxicity is induced in a 37° C. incubator with 5% CO2 in a volume of 100 μl medium supplemented with kainic acid (Wang and Green (2011). J Neurosci. 31(21):7938-7949). Treated and control explants are divided into 3 groups (n=4 for each), and cultured for 5 h, 24 h, and 72 h, respectively. Each of the drugs, amitriptyline, imipramine, LM 22A4 (N,N′,N″Tris(2-hydroxyethyl)-1,3,5-benzenetricarboxamide), THF, DHF, is added at concentrations from 10 nM to 10 μM. Immunohistochemistry is used to identify the pre- and postsynaptic specializations of the organ of Corti.

### Example 4. Screening Compounds for their Effects on SGN Sprouting, Fiber Growth and Synaptogenesis with Hair Cells

In addition to the possibility of protection, the use of Trk agonist drugs to regenerate synapses in vitro is assessed. As shown in FIG. 4, new afferent synapses are generated in explants in the co-cultures (isolated SGNs+denervated organ of Corti) if they are treated with amitriptyline. Each of amitriptyline, imipramine, LM 22A4 (N,N′,N″Tris(2-hydroxyethyl)-1,3,5-benzenetricarboxamide), THF, and DHF is tested for the ability to regenerate afferent synapses. Several doses are explored.

The afferent innervation of hair cells is removed by physical ablation. At postnatal day four to six (P4-P6), the cochlea is dissected and transferred to Petri dishes. The inner and outer hair cells and surrounding supporting cells of the organ of Corti are separated from the SGN at the greater epithelial ridge with a surgical micro-blade, to obtain an intact sensory epithelium devoid of neurons. The de-afferented organ of Corti is then be transferred to a cover glass coated with laminin (25 μg/ml) and poly-L-ornithine (0.01%) in a 4-well Petri dish (Greiner) and maintained overnight at 37° C. in a humidified incubator with 5% CO2 in DMEM/F12, supplemented with N2 and B27 (Gibco). Neurons obtained from newborn mice are added to the de-afferented organ of Corti and cultured for 3-7 days. Formation of new synapses and growth of the fibers to contact hair cells is assessed by immunohistochemistry.

Immunohistochemistry is used to identify the pre- and postsynaptic specializations of the organ of Corti. In the neonatal mouse cochlea, SGN fibers are stained with an antibody against neurofilament and the IHC ribbons can be stained with an antibody against C-terminal-binding protein 2 (CtBP2), a component of ribbon protein, RIBEYE. The postsynaptic densities are stained with an antibody against PSD-95, a membrane associated guanylate kinase (MAGUK) scaffolding protein. Pre- and postsynaptic puncta of CtBP2 and PSD-95 are closely associated at the synaptic zone of the inner hair cells. Thus, PSD-95 should faithfully mark the afferent ribbon synapses between the SGNs and hair cells in the newborn cochlea. Cultures are fixed with 4% paraformaldehyde at room temperature for 20 minutes, followed by permeabilization and blocking with 0.1% Triton-X-100 and 15% normal goat serum for one h. Primary antibodies—anti-CtBP2 (mouse monoclonal IgG1; BD Biosciences), anti-PSD-95 (mouse monoclonal IgG2a, NeuroMab), anti-neurofilament (NF) heavy chain (chicken polyclonal; Chemicon) and anti-myosin VIIa (rabbit polyclonal; Proteus)—are added to the tissue overnight at 4° C. After rinsing three times for ten minutes with 0.01 M PBS, pH 7.4, explants are incubated with secondary antibodies—cyanine-5-conjugated goat anti-mouse IgG1, biotin-conjugated goat anti-mouse IgG2a, Alexa 568-Streptavidin, Alexa Fluor 488 goat anti-chicken or Alexa 647 goat anti-rabbit—for 2 hrs.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or reducing the risk of developing hidden hearing loss (HHL) in a subject, the method comprising administering to the subject a therapeutically effective amount of a small molecule Trk agonist, wherein the method comprises administering one dose up to 12 hours before an episode of noise exposure, and/or optionally one or more doses after the end of the episode of noise exposure.

2. (canceled)

3. The method of claim 1, wherein the small molecule is amitriptyline, imipramine, LM 22A4 (N,N′,N″Tris(2-hydroxyethyl)-1,3,5-benzenetricarboxamide), 7,8-dihydroxyflavone (DHF), 7,8,3′-Trihydroxyflavone (THF), Mab2256, neurotrophin-4 (NT-4), neurotrophin-3 (NT-3), brain derived neurotrophic factor (BDNF), nerve growth factor (NGF), N-acetyl serotonin, N-[2-(5-Hydroxy-1H-indol-3-yl)ethyl]-2-oxo-3-piperidinecarboxamide (HIOC), deoxygedunin, LM-22A4, or tricyclic dimeric peptide 6 (TDP6).

4. The method of claim 1, wherein the small molecule is administered up to 12, 10, 8, 6, 4, 2, or one hour before, or 1-12, 2-12, 2-6, 6-12, or 2-8 hours before, initiation of the noise exposure.

5. The method of claim 1, wherein the small molecule is administered within 0-24 hours after termination of the noise.

6. A method of treating or reducing the risk of hidden hearing loss (HHL) in a subject, the method comprising administering to the subject a therapeutically effective amount of a small molecule therapeutic Trk agonist.

7. (canceled)

8. The method of claim 6, wherein the small molecule is amitriptyline, imipramine, LM 22A4 (N,N′,N″Tris(2-hydroxyethyl)-1,3,5-benzenetricarboxamide), 7, 8-dihydroxyflavone (DHF), 7,8,3′-Trihydroxyflavone (THF), Mab2256, neurotrophin-4 (NT-4), neurotrophin-3 (NT-3), brain derived neurotrophic factor (BDNF), nerve growth factor (NGF), N-acetyl serotonin, N-[2-(5-Hydroxy-1H-indol-3-yl)ethyl]-2-oxo-3-piperidinecarboxamide (HIOC), deoxygedunin, LM-22A4, or tricyclic dimeric peptide 6 (TDP6).

9. The method of claim 6, comprising identifying and/or selecting a subject who has hidden hearing loss.

10. The method of claim 6, wherein identifying and/or selecting a subject who has hidden hearing loss comprises:
measuring a neural-based auditory evoked potential by measuring auditory brainstem response (ABR) or compound action potential (CAP) in a subject;
measuring hair-cell-based responses by measuring distortion product otoacoustic emissions (DPOAE) or summating potentials (SP) in the subject;
and identifying a subject who has a reduced Wave I on ABR or CAP as compared to a normal-hearing subject, and a normal DPOAE or SP, as having HHL.

11. The method of claim 1, wherein the small molecule is administered orally or locally to the ear of the subject.

12. The method of claim 1, wherein the subject is an aging subject or one who will be exposed to noise or ototoxic drugs.

13. The method of claim 12, wherein the exposure is a permanent threshold shifting (PTS) or temporary threshold-shifting (TTS) exposure.

14. The method of claim 1, wherein the small molecule Trk agonist is administered in at least one dose within 6 to 12 or 24 hours after termination of the noise.

15. The method of claim 1, wherein the small molecule Trk agonist is a TrkB and/or TrkC agonist.

16. The method of claim 6, wherein the small molecule Trk agonist is a TrkB and/or TrkC agonist.
